(19) United States
(12) Patent Application Publication
(10) Pub. No.: US 2013/0131532 A1
(43) Pub. Date: May 23, 2013

(54) Medical Diagonstic System

(71) Applicant: Aniekan Umana, Bronx, NY (US)

(72) Inventor: Aniekan Umana, Bronx, NY (US)

(21) Appl. No.: 13/732,477

(22) Filed: Jan. 2, 2013

Related U.S. Application Data
(63) Continuation-in-part of application No. 12/549,441, filed on Aug. 28, 2009, now abandoned.

(51) Int. Cl. A61B 7/04; A61B 7/00

(52) U.S. Cl. CPC A61B 7/04; A61B 7/003
USPC 600/528; 600/586

(57) ABSTRACT

A medical diagnosis system includes recording at least one sound generated from a portion of an internal organ of a patient. Further, the medical diagnosis system includes comparing the at least one sound to a pre-recorded sound of a plurality of pre-recorded sounds for identifying a sound from the at least one sound. Thereafter, the medical diagnosis system includes storing the sound to a memory location based on the identification of the sound from the at least one sound. The sound is stored in form of an electronic file. The electronic file corresponding to the sound is capable of providing diagnostic inputs and a written display corresponding to the diagnosis for a diagnosis of the sound.

### CROSS REFERENCE TO RELATED APPLICATIONS

The present disclosure claims priority on and is a continuation-in-part of under 35 United States Code, Section 120 on the pending U.S. Utility patent application Ser. No. 12/549,441, filed on Aug. 28, 2009, the disclosure of which is incorporated by reference.

### FIELD OF THE DISCLOSURE

The present disclosure relates generally to medical diagnostic systems, and more particularly, to a medical diagnostic system for diagnosing sounds generated from internal organs of a patient.

### BACKGROUND OF THE DISCLOSURE

Often in medical field, a medical practitioner, such as a doctor or a nurse, determines a patient's health by diagnosing sounds generated from internal organs of the patient. Majority of the ailments or diseases of the patient may be detected by diagnosing such sounds generated from the internal organs of the patient. Such sounds may be generated from the internal organs including, but not limited to, heart, lungs, abdomen and artery.

Conventionally, the medical practitioner uses stethoscope to listen to such sounds. However, in order to detect a disease within the body of the patient, the medical practitioner needs to wear the stethoscope over head or ears for listening to the sounds generated from the internal organs of the patient. Further, the medical practitioner needs to listen to the sounds from the internal organs of the patient with full concentration and consciousness for the sounds, which might result into a compromise with accuracy in diagnosing the sounds. Furthermore, such diagnosis of the sounds requires considerable amount of time of the medical practitioner and the patient.

Accordingly, there is a need for a system that is capable of diagnosing sounds generated from internal organs of a patient with enhanced accuracy. Further, the system should be able to be used by a medical practitioner in a convenient and time efficient manner for the diagnosis of the sounds.

### SUMMARY OF THE DISCLOSURE

In view of the foregoing disadvantages inherent in the prior art, the general purpose of the present disclosure is to provide a system for diagnosing sounds generated from internal organs of a patient, configured to include all the advantages of the prior art, and to overcome the drawbacks inherent therein.

Accordingly, an object of the present disclosure is to provide a system for providing diagnosis of sounds generated from internal organs of a patient.

Another object of the present disclosure is to provide a system for providing diagnosis of sounds generated from internal organs of a patient, where the system is comfortable in usage and is not required to be worn over head or ears.

Yet another object of the present disclosure is to provide a system that provides diagnosis of sounds generated from internal organs of a patient with enhanced accuracy and in a time efficient manner.

In light of the above objects, in one aspect of the present disclosure a medical diagnostic system is provided. The medical diagnostic system comprises a recorder, a sound identification module and a memory. The recorder is configured to record at least one sound generated from a portion of an internal organ of a patient. The sound identification module is communicably coupled to the recorder and is configured to compare the at least one sound to a pre-recorded sound of a plurality of pre-recorded sounds (which pre-recorded sounds are preferably stored in the memory of the system) for identifying a sound from the at least one sound. The plurality of pre-recorded sounds are preferably sounds associated with normal and pathological states of at least one human organ or body function. Each of the pre-recorded sounds of the plurality of pre-recorded sounds stored in memory may also include a title or name that corresponds to a sound of the plurality of prerecorded sounds. The memory is communicably coupled to the sound identification module. The memory may store the sound identified by the sound identification module in form of an electronic file.

The electronic file corresponding to the sound identified by the sound identification module is capable of providing diagnostic inputs for a diagnosis of the sound on comparison by the sound identification module of the sound identified by the module (the “recorded sound”) with the pre-recorded sounds stored in the memory. The sound identification module may comprise sound recognition means for comparing the recorded sound with the pre-recorded sounds, and the module may cause the system to display a title or name of a pre-recorded sound with which the sound identification module determines the recorded sound to correspond.

These together with other aspects of the present disclosure, along with the various features of novelty that characterize the present disclosure, are pointed out with particularity in the claims annexed hereto and form a part of this present disclosure. For a better understanding of the present disclosure, its operating advantages, and the specific objects attained by its uses, reference should be made to the accompanying drawing and descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawings, wherein like elements are identified with like symbols, and in which:

FIG. 1 is an exemplary block diagram of a medical diagnostic system for providing diagnosis of sounds generated from internal organs of a patient, according to one embodiment of the present disclosure;

FIG. 2A illustrates an exemplary perspective view of the medical diagnostic system in a flip-open position, according to one embodiment of the present disclosure;

FIG. 2B illustrates an exemplary perspective view of rear side of the medical diagnostic system, according to one embodiment of the present disclosure; and

FIG. 3 illustrates a front perspective view of the medical diagnostic system, according to one embodiment of the present disclosure; and

Like reference numerals refer to like parts throughout the description of several views of the drawings.

### DETAILED DESCRIPTION OF THE DISCLOSURE

The exemplary embodiments described herein detail for illustrative purposes are subject to many variations in composition, structure, and design. It should be emphasized, however, that the present disclosure is not limited to a medical diagnostic system for providing diagnosis of sounds generated from internal organs of a patient, as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting.

The terms “a” and “an” herein do not denote a limitation of quantity, but rather denote the presence of at least one of the referenced items.

The present disclosure provides a medical diagnostic system for diagnosing sounds generated from internal organs of a patient. The medical diagnostic system is configured to record the sounds generated from various portions of the internal organs of the patient. Herein, the internal organs may include, but are not limited to, heart, lungs and abdomen. Further, the medical diagnostic system of the present disclosure is also capable of providing diagnosis of bruit sounds from any artery of the patient. The bruit sound may occur due to obstruction faced by blood due to blockage in the artery. For the purpose of this description, artery is also considered as an internal organ of the patient. Diagnosis of the sounds from the internal organs may help in detecting any disease or ailment in the body of the patient. The medical diagnostic system may detect and store such sounds from the internal organs at a memory location. Further, various attributes of the stored sound may be displayed, printed or used for future diagnosis or references by a medical practitioner, such as may be a doctor, a nurse or the patient himself.

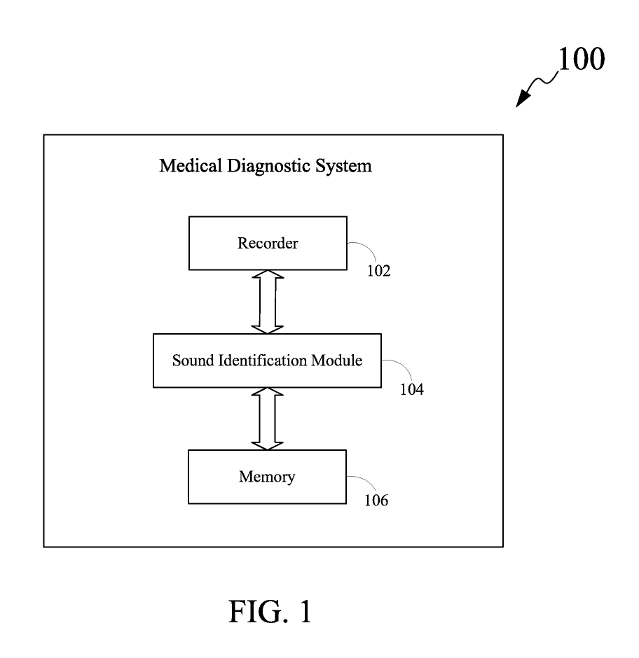

FIG. 1 is an exemplary block diagram of a medical diagnostic system 100, according to one embodiment of the present disclosure. The medical diagnostic system 100 (hereinafter referred to as “the system 100”) may be used for diagnosing sounds generated from the internal organs of the patient in order to determine patient's health.

The system 100 includes a recorder 102, a sound identification module 104 communicably coupled to the recorder 102 and a memory 106 communicably coupled to the sound identification module 104. The system 100 may be placed over the body of patient above an internal organ for diagnosing the sound generated from the internal organ of the patient. More specifically, the system 100 may be placed over the body of the patient by aligning the system 100 above a portion of the internal organ, which sounds needs to be diagnosed.

The recorder 102 is configured to record or read (hereinafter referred to as “record”) sounds from various portions of the internal organ of the patient. The recorder 102 may record the sounds by press of a button (not shown) of the system 100. The system 100 may include a plurality of buttons (not shown) corresponding to various portions of internal organs of the patient. Each button may be used to record a sound from a particular portion of a particular internal organ of the patient. For example, the heart may have portions including, but not limited to, an aortic valve, a pulmonary artery valve, a tricuspid valve and a mitral valve. Accordingly, there may be separate buttons to actuate the recorder 102 to record sounds from the portions of the heart. In one embodiment of the present disclosure, there may be four separate buttons for recording the sounds generated from the four portions of the heart.

Similarly, the lungs may include portions including, but not limited to, anterior chest wall, posterior chest wall, right base area, and left base area. Further, the abdomen may have four portions including, but not limited to, right flank, left flank, cardia and umbilicus. Further, the bruit sound may also be recorded from arteries at various portions within the body of the patient, such as carotid arteries, renal arteries at flank, abdominal aorta at cardia region of the abdomen, and around umbilicus portion. The recorder 102 may be a high sensitive digital recording device, which is capable of detecting and recording the sounds. The recorder 102 will record the sounds from the various portions of the heart, the lungs, the abdomen and the arteries. In one embodiment, the recorder 102 may include four separate microchips based digital recording devices for recording sounds generated from the heart, lungs, abdomen and arteries of the patient. The recorder 102 is configured to record sounds generated from a portion of an internal organ when a button corresponding to the portion is actuated. Further, the recorder 102 is also configured to record multiple sounds for a single portion of an internal organ. For this, the system 100 may be equipped with a special button to record multiple instances of the sounds from the single portion of the internal organ.

In a typical application of the system 100 for diagnosis of an internal organ, the recorder 102 records at least one sound from a portion of the internal organ of a subject or patient. The at least one sound recorded by the recorder 102 is hereinafter referred to as “recorded sound(s)”. Further, the sound identification module 104 may identify a sound from the recorded sound(s) as a reading of the sound for the portion of the internal organ. The sound identification module 104 may identify the sound as the reading by comparing the recorded sound(s) to a plurality of pre-recorded sounds by way of sound recognition means. The sound recognition means of the sound identification module may comprise any known means for comparing or identifying sounds, such as Dragon™ or Microsoft® voice recognition software.

The identification of the sound as the reading from the recorded sound(s) for the portion of the internal organ is based on the plurality of pre-recorded sounds. The plurality of pre-recorded sounds may be stored in the memory 106 or may be accessed by the system 100 from an external source. Each of the plurality of pre-recorded sounds may be related to a particular portion of a particular internal organ of the patient or a particular body function or condition of the patient. More specifically, a pre-recorded sound may be a sample sound corresponding to a particular portion of an internal organ. It will be apparent to a person skilled in the art that the plurality of pre-recorded sounds may be stored in the memory 106 before using the system 100 for diagnosing the sounds from the internal organ of the patient. The sound identification module 104 is configured to compare the recorded sound(s) to a pre-recorded sound of the plurality of pre-recording sounds. In one embodiment of the present disclosure, the sound identification module 104 may be configured to identify multiple sounds from the recorded sound(s) as the readings for the portion of the internal organ.

The sound identification module 104 may utilize the sound recognition means for comparing the recorded sound (before or after identification thereof) with the pre-recorded sounds, as it will be apparent that the sound recognition means of the sound identification module may be capable of comparing sounds as well as identifying sounds. On comparison of the recorded sound with the pre-recorded sounds, the system 100 may display an output on a display screen, which display will be described in more detail below.

Further, the sound identified by the sound identification module 104 may be stored to a location in the memory 106 of the system 100. The sound is stored in the memory 106 in form of an electronic file. The electronic file is capable of providing diagnostic inputs for a diagnosis of the sound to the medical practitioner, which inputs may be generated on comparison of the recorded sound with the pre-recorded sounds, for example. In an embodiment of the present disclosure, the electronic file may include various attributes of the sound that may provide the diagnostic inputs, such as a visual attribute or a printable attribute, an audible attribute, and the like. Herein, the visual attribute of the sound may refer to a written description of the diagnostic inputs. For instance, in the event that the recorded sound is retrieved from a subject's chest area and is compared with a pre-recorded sound and identified accordingly, the visual attribute may comprise “RALES” or “RHONCHI” to indicate the diagnosis of the subject. Further, the printable attribute of the sound may be printed on a physical media. The printable attribute of the sound may represent the sound on the physical media upon printing, which may provide the diagnostic inputs to the medical practitioner for the diagnosis of the sound. Similarly, the audible attribute of the sound will allow the sound to be heard by the medical practitioner. Further, the electronic file may also include other attributes of the sound, which will allow the sound to be transmitted to an external destination, or shared between the system 100 and an external device.

It would be apparent to those ordinary skilled in the art that the system 100 is capable of replaying, printing, transmitting, saving or sharing the readings of the sound corresponding to the portion of the internal organ of the patient. Accordingly, the system 100 may provide an accurate record of the readings of the sounds from various portions of the internal organ for the diagnosis by the medical practitioner, or for future references of the readings of the sounds.

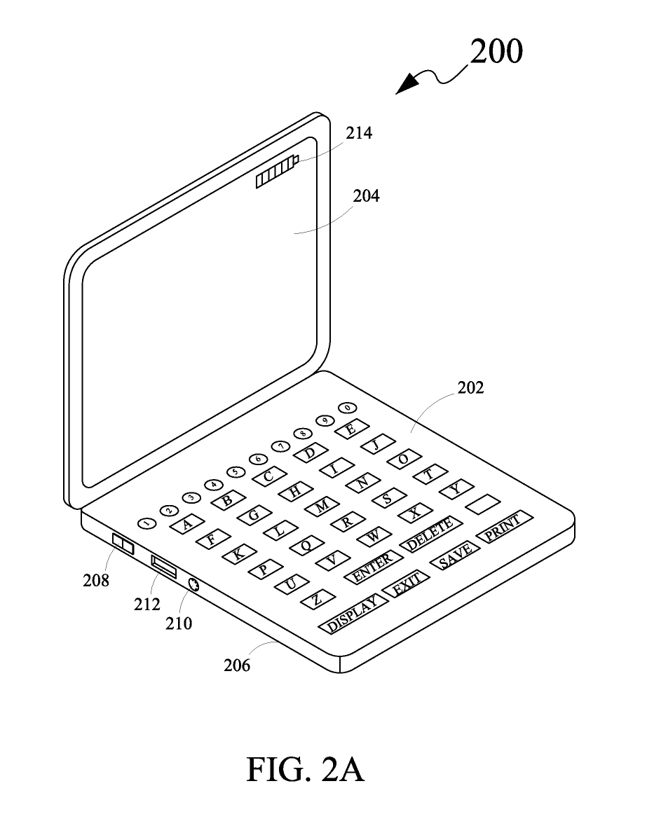

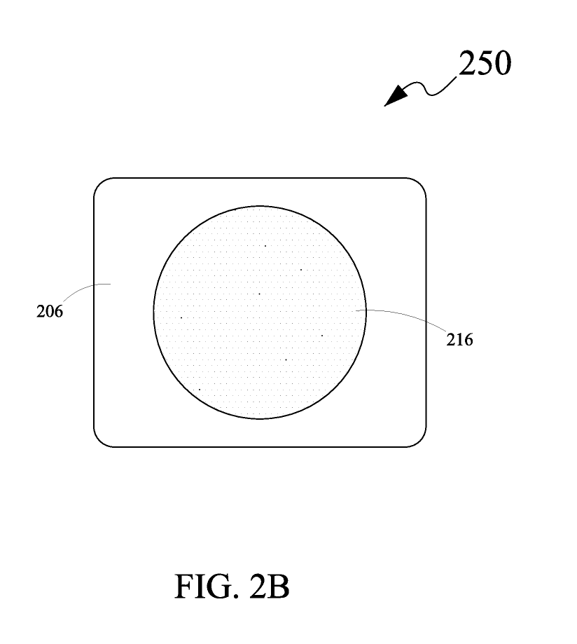

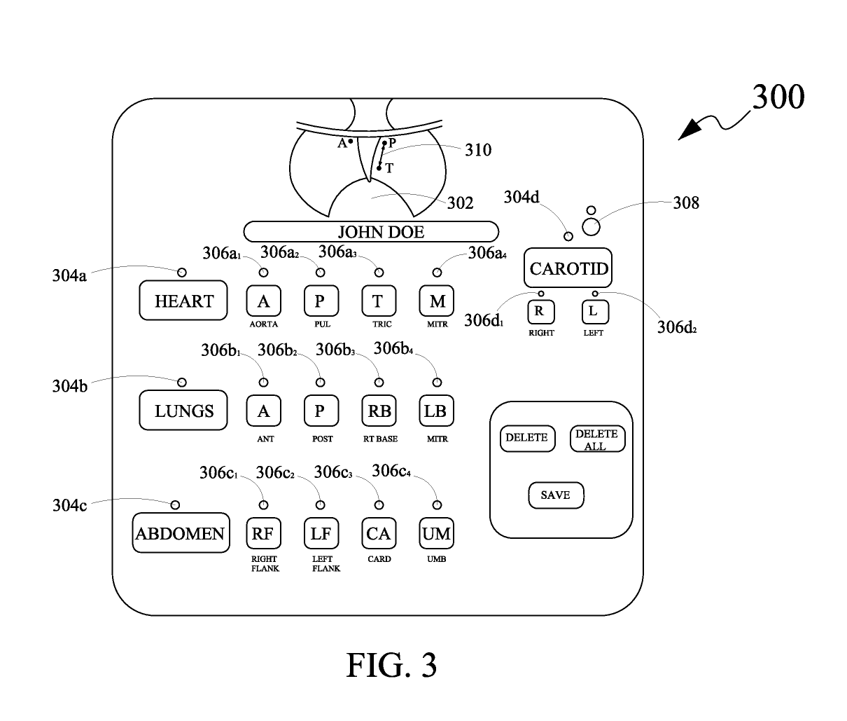

Physical representations of the system 100 and its components are shown in FIGS. 2 and 3, embodying the present disclosure. The system 100 will further be described herein in conjunction with FIGS. 2A, 2B and 3.

Referring now to FIG. 2A, an exemplary perspective view of the system 100 is shown, according to one embodiment of the present disclosure. The system 100 may look like a regular cell phone or a flip-open Personal Digital Assistance (PDA) device. However, it will be apparent to a person skilled in the art that the system 100 may be of various shapes and designs. Further, it would be apparent to those skilled in the art that the system 100 is portable and the medical practitioner may wear the system 100 on his/her belt, pocket, and the like. Furthermore, the system 100 is not required to be worn over head or ears as opposed to conventional stethoscopes. The FIG. 2A illustrates the system 100 in a flip open position that shows a flip-open perspective view 200 of the system 100.

The flip-open perspective view 200 of the system 100 represents a key area 202 and a display screen 204. The key area 202 includes various keys to type in name of a patient. Further, an enter key (shown in the key area 202) may be pressed after entering the name of the patient by using the key area 202. The system 100 includes a base 206 of the key area 202. The system 100 includes an on/off button 208, a battery charging port 210 and a Universal Serial Bus (USB) port 212 placed on a side of the base 206. The display screen 204 includes a battery indicator 214 for the system 100.

The display screen 204 will be communicably coupled to the memory 106. The display screen 204 is configured to display the display attributes corresponding to the sound. By seeing the display attributes corresponding to the sound on the display screen 204, the medical practitioner may review the display screen to observe or confirm a diagnosis of the sound. The on/off button 208 is used to switch ON and switch OFF the system 100. The battery charging port 210 is used for charging the system 100. The battery charging port 210 may be connected to an external power source through a suitable adaptor. Further, the USB port 212 is utilized for connecting the memory 106 of the system 100 to a peripheral device (not shown). The USB port 212 may be used to connect the system 100 to the peripheral device, such as a printer. The printer may be utilized by pressing a “print” key provided in the key area 202. Other examples of the peripheral device may include, but are not limited to, a personal computer, a facsimile and a photocopier machine. Accordingly, the medical practitioner may access the sound through the peripheral device for the diagnosis of the sound.

The recorder 102 may be configured on a rear side of the base 206. The recorder 102 includes a hypersensitive diaphragm microphone configured to tap the sounds generated from various portions of the internal organs, shown in a perspective view 250 of rear side of the system 100 in FIG. 2B. Referring now to FIG. 2B, the base 206 representing a hypersensitive diaphragm microphone 216 is shown, in accordance with an embodiment of the present disclosure. The hypersensitive diaphragm microphone 216 may be a sensitive digital recording device. The hypersensitive diaphragm microphone 216 may be placed on the portion of the internal organ of the patient to record at least one sound generated from the portion. Further, for using the system 100 for diagnosing the sound, the system 100 may be flipped off and a front cover of the system 100 is used, which is described in conjunction with FIG. 3.

Referring now to FIG. 3, a front perspective view illustrating a front cover 300 of the system 100 is shown. The front cover 300 of the system 100 is utilized for diagnosing sounds generated from a portion of an internal organ of a patient. The name of the patient entered from inside region of the system 100, i.e., from the key area 202, may be shown on the front cover 300. For example, the name of the patient “JOHN DOE” is shown in FIG. 3. The front cover 300 includes a diagnostic window 302 and a plurality of buttons corresponding to internal organs of the patient. As shown in FIG. 3, buttons 304a, 304b, 304c, 304d are used for the various internal organs of the patient, for example, button 304a may be used for diagnosing sounds generated from the heart of the patient. Similarly, buttons 304b, 304c and 304d may be used for lungs, abdomen and artery, respectively, of the patient. Hereinafter, the buttons (304a, 304b, 304c and 304d) for the internal organs of the patient will be collectively referred to as “the buttons 304”. In an embodiment of the present disclosure, the buttons 304a, 304b, 304c, 304d may also act as light indicators, which upon activation indicate the diagnosis of sounds generated from their corresponding portion.

Further, each of the buttons 304 may also be provided with a set of buttons for diagnosing different portions of their corresponding internal organ. Each internal organ of the patient may have different portions and the sounds generated from these portions may be recorded by (de)pressing their corresponding buttons shown on the front cover 300 of the system 100. For example, the heart may have four portions such as a portion related to aortic valve (hereinafter referred to as “aortic valve”), a portion related to pulmonary artery valve (hereinafter referred to as “pulmonary artery valve”), a portion related to tricuspid valve (hereinafter referred to as “tricuspid valve”) and a portion related to mitral valve (hereinafter referred to as “mitral valve”). For each portion of the heart, the front cover 300 may have a separate button, for example, a button 306a1 (represented by “A”) corresponds to the aortic valve of the heart. Similarly, a button 306a2 (represented by “P”), a button 306a3 (represented by “T”) and a button 306a4 (represented by “M”) correspond to the pulmonary artery valve, the tricuspid valve and the mitral valve of the heart, respectively.

Further, the lungs may also include four portions. The portions of the lungs may include a portion of anterior chest wall (hereinafter referred to as “anterior chest wall”), a portion of a posterior chest wall (hereinafter referred to as “posterior chest wall”), a portion of a right base area (hereinafter referred to as “right base area”) and a portion of a left base area (hereinafter referred to as “left base area”). For these portions of the lungs, the front cover 300 may have separate buttons, for example, a button 306b1 (represented by “A”) for the anterior chest wall of the lungs. Similarly, a button 306b2 (represented by “P”), a button 306b3 (represented by “RB”) and a button 306b4 (represented by “LB”) are used for the posterior chest wall, the right base area and the left base area of the lungs, respectively.

Furthermore, the abdomen may include four portions, such as right flank, left flank, cardia, and umbilicus. The front cover 300 may have separate buttons such as a button 306c1 (represented by “RF”) for the right flank, a button 306c2 (represented by “LF”) for left flank, a button 306c3 (represented by “CA”) for the cardia and a button 306c4 (represented by “UM”) for the umbilicus. In addition to this, the front cover 300 may have buttons 306d1 and 306d2 for the arteries, such as for right and left carotid of the patient. Hereinafter, the buttons for different portions of the internal organs of the patient will be collectively referred to as ‘the buttons 306’.

The front cover 300 may be used to initiate the diagnosis by pressing any button on the front cover 300. In an embodiment of the present disclosure, an indicator, such as a green light may be turned on, which indicates that the button has been activated. The medical practitioner may press a button from the buttons 304 and/or the buttons 306 for recording at least one sound generated from a portion of an internal organ corresponding to the pressed button. Further, the at least one recorded sound (hereinafter recorded sound(s)) may be compared against a pre-recorded sound by the sound identification module 104 of the system 100 to identify a sound as a reading for the diagnosis. Further, the recorded sound is stored in a memory, such as the memory 106. The sound identification module 104 is not visible in FIGS. 2 and 3, however it would be apparent to those skilled in the art that the sound identification module 104 may be fitted inside the base 206 of the system 100. In an embodiment of the present disclosure, the sound identification module 104 includes a sound recognition means, which sound recognition means may be a microchip based device, incorporating microchip processors. The microchip processors will be configured to identify the sound from the recorded sound(s) and will send the sound for storing in the memory 106.

Further, the front cover 300 may include a special button such as a button 308 for taking multiple recordings of sounds at one portion of an internal organ. For example, if the medical practitioner wants to take multiple recordings of the posterior part of the lungs, the medical practitioner may press the “P” button for the posterior part and then press the button 308. In this way, the user may take multiple recordings that may be displayed on the diagnostic window 302. In one embodiment, if the medical practitioner wants to take multiple readings of sounds between one portion to another portion of an internal organ, the user may press the button 308 in unison with buttons related to both portions. For example, the medical practitioner may wish to take recordings along left sternal border, the medical practitioner may press the “T” button, and the button 308. Thereafter, the base 206 may be moved along the left sternal border, as shown by an arrow 310. In this manner, the medical practitioner will be taking multiple readings along the arrow 310.

Further, the front cover 300 may include other buttons for commands like “delete”, “delete all” and “save” for deleting and saving the readings of the sounds. The system 100 may produce a beep to indicate that the system 100 has completed its task of saving or deleting the readings. The medical practitioner may then move the base 206 to take further readings of a next portion and press the corresponding button for diagnosing the sound generated from the next portion. Further, the medical practitioner may wish to perform various functions related to the detected sound such as display, save or print. These functions have already been explained in conjunction with FIGS. 2A and 2B.

It would be apparent to those skilled in the art that various portions of the internal organs, such as heart, lungs, abdomen and artery may generate different sounds, which may be recorded with the press of their corresponding buttons. For example, a portion related to aortic valve of the heart (that may be accessed through button 306a1 (“A”) of the front cover 300 of the system 100, as shown in FIG. 3) may record sounds such as a systolic murmur, diastolic murmur, increased S₂ sound, ejection click and decreased S₂ sound. Similarly, the pulmonary artery valve of the heart (that may be accessed through button 306a1 (“P”) of the front cover 300, as shown in FIG. 3) may record sounds including, but not limited to, a systolic murmur, diastolic murmur, increased S₂ sound, ejection click, decreased S₂ sound and continuous rumbling murmur. The tricuspid valve (that may be accessed through “T” button of the front cover 300, as shown in FIG. 3) may record sounds including, but not limited to, systolic murmur, diastolic murmur, increased S₁ sound, and decreased S₁ sound. The mitral valve section of the heart (that may be accessed through “M” button of the front cover 300, as shown in FIG. 3) may record sounds including, but not limited to, a systolic murmur, diastolic murmur, S₁ sound, opening snap, S₃ sound, S₄ sound, gallop sound, splitting of S₁ sound and decreased S₁ sound.

Similar to the different portions of the heart, different portions of the lungs may also generate different sounds, which may be recorded with the press of their corresponding buttons. For example, the anterior chest wall (that may be accessed through “A” button of the front cover 300 shown in FIG. 3) may include sounds such as vesicular sound, bronchovesicular sound, bronchial sound, rales, rhonchi and friction rub. The other portions of the lungs, such as the posterior chest wall (that may be accessed through “P” button of the front cover 300 shown in FIG. 3), the right base area (that may be accessed through “RB” button of the front cover 300 shown in FIG. 3) and the left base area (that may be accessed through “LB” button of the front cover 300 shown in FIG. 3) may record sounds similarly as the sounds recorded by button related to the anterior chest wall section.

Further, the buttons related to the abdomen, when pressed, may record various sounds such as normal borborygmi, increased borborygmi (hyperactivity), decreased borborygmi (hypoctivity), and no borborygmi (iLeus). Additionally, buttons related to the artery may be utilized to record bruit sounds generated from various arteries of the patient. The bruit sounds may be recorded from carotid arteries at the neck, renal arteries at the flank, abdominal aorta at the cardia region of the abdomen, and around the umbilicus area.

The electronic file is capable of providing diagnostic inputs for a diagnosis of the sound to the medical practitioner. The electronic file corresponding to the sound may include various attributes of the sound, such as a visual attribute, a printable attribute, an audible attribute, and the like. The visual attribute of the sound may be displayed on a display screen, such as the display screen 204. A medical practitioner may further diagnose the sound by seeing the visual attribute of the sound. Herein, the visual attribute may include a written description of the diagnostic inputs, which may provide the diagnostic inputs to the medical practitioner for the diagnosis of the sound. Further, a printable attribute of the sound may be printed on a physical media. Furthermore, an audible attribute of the sound may be heard by the medical practitioner and utilized as a reference in observing or confirming a diagnosis that is shown on the display screen.

As described above, the present disclosure describes a medical diagnostic system for providing diagnosis of sounds generated from internal organs of a patient. The various embodiments of the present disclosure may be utilized for providing assistance to a medical practitioner in determining the health of the patient by diagnosing these sounds. Such diagnostic sounds may be stored in the medical diagnostic system that may be analyzed as per the convenience of the medical practitioner. Accordingly, the medical diagnostic system may reduce the chances of wrong diagnosis of the sounds. Further, the medical diagnostic system may help the medical practitioner to look after many patients at a time, as there is provision of storing the sounds of each patient in the medical diagnostic system. Further, the system facilitates diagnosing sounds in a more efficient manner.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present disclosure.

What is claimed is:

1. A medical diagnostic system, comprising:
a recorder configured to record at least one sound generated from an internal organ of a patient;
a sound identification module communicably coupled to the recorder, the sound identification module comprising a sound recognition means, which sound recognition means is configured to compare the at least one sound to a pre-recorded sound of a plurality of pre-recorded sounds for identifying a sound from the at least one sound; and
a memory communicably coupled to the sound identification module, the memory configured to store the sound recorded by the recorder and identified by the sound identification module in form of an electronic file, wherein the electronic file corresponding to the sound may generate diagnostic inputs for a diagnosis of the sound based on the comparison of the at least one sound generated from an internal organ of a patient and a pre-recorded sound of the plurality of prerecorded sounds.

2. The medical diagnostic system of claim 1, further comprising a display screen for displaying a visual attribute corresponding to the sound, the visual attribute corresponding to the sound stored in the electronic file, wherein the visual attribute provides the diagnostic inputs for the diagnosis of the sound, which diagnostic inputs may comprise at least display of a word that that corresponds to the diagnosis of the sound.

3. The medical diagnostic system of claim 1, wherein the electronic file comprises a printable attribute of the sound, the printable attribute of the sound capable of being printed on a physical media and is capable of providing the diagnostic inputs for the diagnosis of the sound.

4. The medical diagnostic system of claim 1, wherein the electronic file comprises an audible attribute of the sound, the audible attribute of the sound capable of being heard by a medical practitioner and is capable of providing the diagnostic inputs for the diagnosis of the sound.

5. The medical diagnostic system of claim 1, wherein the memory is configured to store a name of the patient.

6. The medical diagnostic system of claim 1 further comprising a Universal Serial Bus (USB) port, the USB port configured to connect the memory to a peripheral device for providing access of the electronic file to the peripheral device.

7. The medical diagnostic system of claim 1, wherein the internal organ comprises one of a heart of the patient, lungs of the patient, an abdomen of the patient and at least one artery of the patient.
